(19) United States
(12) Patent Application Publication
(10) Pub. No.: US 2023/0363652 A1
(43) Pub. Date: Nov. 16, 2023

(54) Intravascular Pressure Sensing Using Inner Sheath

(71) Applicant: CANON U.S.A., INC., Melville, NY (US)

(72) Inventors: Lampros Athanasiou, Medford, MA (US); Mark Alan Hamm, Lynnfield, MA (US); Christopher Douglas Brushett, Cambridge, MA (US)

(21) Appl. No.: 17/744,121

(22) Filed: May 13, 2022

(51) Int. Cl. A61B 5/0215; A61B 1/00; A61B 1/005; A61B 1/313; A61B 1/06; A61B 5/029; A61B 5/00

(52) U.S. Cl. CPC A61B 5/0215; A61B 1/00135; A61B 1/005; A61B 1/3137; A61B 1/0623; A61B 5/029; A61B 5/0066; A61B 1/00006; A61B 1/00045

(57) ABSTRACT

A catheter comprises an imaging core, an inner sheath enclosing the imaging core, an outer sheath surrounding the inner sheath, and a flexible membrane arranged on the outer sheath and configured to deflect in response to intravascular pressure. At least part of the inner sheath and part of the outer sheath are nested within each other. A chamber is defined by the membrane, and the parts of the inner and outer sheaths that are nested within each other. The chamber provides a space into which the membrane is deflected when surrounded by fluids. A processor controls the imaging core to acquire pressure data by scanning the membrane with light transmitted through the chamber, and to acquire image data by scanning a vessel wall with light transmitted through the inner sheath. If the membrane breaks, the chamber prevents fluids from entering the imaging core, and the catheter continues acquiring image data.

### CROSS-REFERENCE TO RELATED APPLICATIONS

n/a

### BACKGROUND INFORMATION

### Field of Disclosure

The present disclosure generally relates to medical devices. More particularly, the disclosure is directed to an intravascular pressure sensing sheath that can be used in a hybrid catheter for intravascular imaging and pressure sensing.

### Description of Related Art

Coronary artery disease (CAD) is a type of heart disease that develops when the arteries of the heart cannot deliver enough oxygen-rich blood to the heart. CAD causes the reduction of blood flow to the heart muscle due to build-up of plaque (atherosclerosis) and the resultant narrowing (stenosis) of the arteries. Modern technologies provide several minimally invasive surgical (MIS) tools used in percutaneous coronary interventions (PCI) to diagnose and treat patients suffering of CAD. The most popular PCI technique for diagnosing CAD and restoring blood flow to the heart is coronary angioplasty. Angioplasty relies on thin catheters inserted into the vascular system percutaneously through an artery in the wrist or leg of a patient. Image guidance using ultrasound (US), magnetic resonance imaging (MRI), and/or X-ray fluoroscopy combined with radiopaque contrast dye injected into the bloodstream serves to guide the catheters to the region of the body to be treated. To treat a narrowing in a blood vessel, a guidewire is passed through the stenosis in the vessel, and a balloon on a catheter is passed over the guidewire and into the desired position while the physician observes the procedure on a display screen. The positioning of the balloon is verified by the chosen type of image guidance (fluoroscopy, ultrasound, MRI, or other), and the balloon is inflated using a fluid (e.g., water, air, and/or a contrast dye) to re-open (expand) the vessel occlusion. In some cases, a stent may be installed in the place of the vessel occlusion depending on the severity of the stenosis. At the conclusion of the procedure, the balloon, guidewire and catheter are removed.

Prior to, during, and after performing a percutaneous interventional procedure such as angioplasty, measuring the intracoronary pressure is critical. Intracoronary pressure is a procedure that measures the blood pressure inside the coronary arteries, and is used to estimate how the stenosis is affecting the blood flow of the vessel. Intracoronary pressure provides the pressure differences across a stenosis and determines the likelihood that the stenosis impedes oxygen delivery to the heart muscle. Towards this purpose, intravascular guidewire or catheter devices are conventionally developed with sensors which measure the blood pressure inside the vessels. These devices use an electrical or optical or electro-optical sensor attached to an intravascular shaft (guidewire or catheter) to measure exclusively the pressure in the areas of interest inside the vessel.

A currently accepted technique for assessing the severity of stenosis in a blood vessel, including ischemia causing lesions, is fractional flow reserve (FFR). FFR is a calculation of the ratio of a distal pressure measurement (taken on the distal side of the stenosis) relative to a proximal pressure measurement (taken on the proximal side of the stenosis). FFR provides an index of stenosis severity that allows a determination as to whether the blockage limits blood flow within the vessel to an extent that PCI treatment is required. The normal value of FFR in a healthy vessel is 1.00, while values less than about 0.80 are generally deemed of significant concern to require PCI treatment.

In the current state of the art, dedicated intravascular catheters and/or guidewires with sensors are widely utilized to measure the pressure within a blood vessel. See, for example, U.S. Pat. Nos. 10,932,670 B2, 10,307,070 B2, 10,130,269 B2, 8,715,200 B2, 6,106,476, 5,902,248, and pre-grant patent application publications US 2015/0367105 A1 and US 2019/0343409 A1, among others. In particular, U.S. Pat. No. 10,932,670B2 discloses an OCT imaging system that uses a catheter to interchangeably generate images of a vascular lumen and measure intravascular pressure. Here, for pressure detection, the catheter system uses reflections from a rotating imaging core to detect the distance from the imaging core to a flexible membrane arranged on the sheath of the catheter. The catheter detects intravascular pressure, by measuring the distance from the imaging core to the deformed membrane. An interface medium at the distal end of the optical fiber is used as a reference to measure the distance from the imaging core to the deformed membrane. This system is likely to give inaccurate results due to non-uniform rotational distortion (NURD) caused by the rotation of the imaging core with respect to the flexible membrane arranged on the catheter sheath. In addition, if the membrane fails (e.g., the membrane might break or puncture since it is very thin), intravascular fluids will enter the imaging core, and the catheter will lose its intended use (e.g., imaging and pressure measurement).

Therefore, although the use of catheters and/or guidewires with pressure sensors is widely known, there remains a need for improved intravascular devices, systems, and methods which would allow for safer and more accurate pressure measurements and image data collection with the same intravascular imaging catheter.

### SUMMARY OF EXEMPLARY EMBODIMENTS

One or more embodiments of the present disclosure are directed to an improved intravascular imaging catheter configured for accurate pressure measurements and fast image data collection from a patient's vascular lumen in a single pullback procedure.

According to at least one embodiment of the present disclosure, one or more of limitations of conventional catheters are addressed by a new catheter design which leverages the imaging signal of an imaging catheter to directly measure intravascular pressure and acquire images of the vessel stenosis. The novel catheter device is an intravascular imaging catheter having a novel diaphragm-based pressure sensor, and nested inner and outer catheter sheaths. In one embodiment, a flexible membrane is attached to a surface of the outer sheath, and an imaging core is arranged inside the inner sheath. The inner sheath is nested inside the outer sheath, such that the two sheaths are concentric to each other and at a predetermined distance therebetween. During intravascular intervention, the catheter first scans the diaphragm-based pressure sensor, and subsequently scans the vessel wall in a single pullback pass. A computer processor uses data collected from the scanned diaphragm to calculate intravascular pressure, and uses data collected from the scanned vessel wall to generate one or more images of the vessel wall. In the event that the diaphragm-based pressure sensor fails (e.g., the diaphragm breaks), the inner sheath protects the imaging core from intravascular fluids, so that the catheter can continue to be used for imaging purposes.

In one embodiment, the catheter is an optical coherence tomography (OCT) catheter having an outer sheath, an inner sheath, and an imaging core arranged substantially coaxial to each other. The imaging core includes an optical fiber configured to rotate inside the inner sheath, and the outer sheath includes a flexible membrane which bends in response to pressure from intravascular fluids. In this embodiment, during a pullback procedure, light from the optical fiber distal end first scans the diaphragm through the inner sheath, and subsequently scans the inner surface of the vessel through the inner sheath. The reflected light is collected by the OCT system producing a first OCT image which depicts the diaphragm surface, and a second OCT image which depicts the vessel surface. The diaphragm is implemented by a thin and flexible membrane. The sensitivity of the diaphragm allows small pressure changes (e.g., in small amounts of ±5 mm Hg) to deform the flexible membrane. The OCT image of the membrane depicts the diaphragm deformation as a difference in position of the membrane before and after the pressure change. A processor calculates an amount of deflection of membrane using the inner sheath as a reference. Then, the processor can calculate the intravascular pressure by correlating the amount of deflection of diaphragm to fractional flow reserve (FFR) or other intravascular pressure parameters. Moreover, the system can calculate the pressure changes between two points of interest (e.g., distal and proximal to stenosis), and therefore it is possible to assess the physiological significance of CAD during percutaneous coronary intervention (PCI).

According to another embodiment, a multifunction catheter system, comprising: an outer sheath having a proximal end and a distal end with a lumen extending therethrough along a longitudinal axis thereof, the outer sheath configured for insertion into a vessel in the vasculature of a patient; a pressure sensor having a flexible membrane that deflects in response to intravascular pressure; an inner sheath having an outer diameter configured for insertion into the lumen of the outer sheath; an imaging core arranged inside the inner sheath and configured to scan the inner sheath and the flexible membrane, and subsequently scan the vessel wall with a beam of radiative energy directed at an angle with respect to the longitudinal axis; and a processor configured to determine an amount of intravascular pressure based on radiative energy reflected or backscattered by the inner sheath and by the flexible membrane, and to generate an image of the vessel based on radiative energy reflected or backscattered by the vessel wall.

These and other objectives, features, and advantages of the present disclosure will become apparent upon reading the following detailed description of exemplary embodiments of the present disclosure, when taken in conjunction with the appended drawings, and provided claims.

### BRIEF DESCRIPTION OF DRAWINGS

FIG. 1 is a diagram of an OCT imaging system 100 having a catheter 160 configured to obtain pressure data and image data from a vessel 170, according to the present disclosure;

FIG. 2 illustrates a first embodiment of the catheter 160 inserted into a vessel 170;

FIG. 3 shows an embodiment of the catheter 160 comprising a pressure sensor 180;

FIG. 4 illustrates an exemplary longitudinal view of catheter 160 undergoing a data collection operation;

FIG. 5A, FIG. 5B, and FIG. 5C illustrate various views of a first embodiment of the pressure sensor 180 useful for monitoring blood pressure and/or FFR in a vessel;

FIG. 6A shows a top view along the longitudinal direction of the catheter (a view in the lengthwise direction parallel to the catheter axis Ox) of the pressure sensor 180. FIG. 6B shows the working principle of the pressure sensor 180;

FIG. 7 show an exemplary algorithm of a process for acquiring pressure data and image data from a lumen using the OCT catheter 160, and generating an image of the lumen and calculating a pressure parameter of the lumen (e.g., a vessel);

FIG. 8A, FIG. 8B, and FIG. 8C illustrate various views of a second embodiment of the pressure sensor 180 useful for monitoring blood pressure and/or FFR in a vessel.

### DETAILED DESCRIPTION OF EXEMPLARY EMBODIMENTS

Before the various embodiments are described in further detail, it is to be understood that the present disclosure is not limited to any particular embodiment. It is also to be understood that the terminology used herein is for the purpose of describing exemplary embodiments only, and is not intended to be limiting.

Various objectives, features and advantages of the present disclosure will become apparent from the following detailed description when taken in conjunction with the accompanying figures showing illustrative embodiments of the present disclosure. Throughout the figures, the same reference numerals and characters, unless otherwise stated, are used to denote like features, elements, components or portions of the illustrated embodiments. In addition, while the subject disclosure is described in detail with reference to the enclosed figures, it is done so in connection with illustrative exemplary embodiments. It is intended that changes and modifications can be made to the described exemplary embodiments without departing from the true scope of the subject disclosure as defined by the appended claims. Although the drawings represent some possible configurations and approaches, the drawings are not necessarily to scale and certain features may be exaggerated, removed, or partially sectioned to better illustrate and explain certain aspects of the present disclosure. The descriptions set forth herein are not intended to be exhaustive or otherwise limit or restrict the claims to the precise forms and configurations shown in the drawings and disclosed in the following detailed description.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached”, “coupled” or the like to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown in one embodiment can apply to other embodiments. It will also be appreciated by those of skill in the art that references to a structure or feature that is disposed “adjacent” to another feature may have portions that overlap or underlie the adjacent feature.

The terms first, second, third, etc. may be used herein to describe various elements, components, regions, parts and/or sections. It should be understood that these elements, components, regions, parts and/or sections are not limited by these terms of designation. These terms of designation have been used only to distinguish one element, component, region, part, or section from another region, part, or section. Thus, a first element, component, region, part, or section discussed below could be termed a second element, component, region, part, or section merely for purposes of distinction but without limitation and without departing from structural or functional meaning. The terms “in sequence”, “sequential”, “sequentially”, and variations thereof are meant to describe a given order which progresses by one from each element to the next in order of succession.

As used herein, the singular forms “a”, “an”, and “the” are intended to include the plural forms as well, unless the context clearly indicates otherwise. It should be further understood that the terms “includes” and/or “including”, “comprises” and/or “comprising”, “consists” and/or “consisting” when used in the present specification and claims, specify the presence of stated features, integers, steps, operations, elements, and/or components, but do not preclude the presence or addition of one or more other features, integers, steps, operations, elements, components, and/or groups thereof not explicitly stated. Further, in the present disclosure, the transitional phrase “consisting of” excludes any element, step, or component not specified in the claim. It is further noted that some claims or some features of a claim may be drafted to exclude any optional element; such claims may use exclusive terminology as “solely,” “only” and the like in connection with the recitation of claim elements, or it may use of a “negative” limitation.

The term “about” or “approximately” as used herein means, for example, within 10%, within 5%, or less. In some embodiments, the term “about” may mean within measurement error. In this regard, where described or claimed, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range, if recited herein, is intended to be inclusive of end values and includes all sub-ranges subsumed therein, unless specifically stated otherwise. As used herein, the term “substantially” is meant to allow for deviations from the descriptor that do not negatively affect the intended purpose. For example, deviations that are from limitations in measurements, differences within manufacture tolerance, or variations of less than 5% can be considered within the scope of substantially the same. The specified descriptor can be an absolute value (e.g. substantially spherical, substantially perpendicular, substantially concentric, etc.) or a relative term (e.g. substantially similar, substantially the same, etc.).

Unless specifically stated otherwise, as apparent from the following disclosure, it is understood that, throughout the disclosure, discussions using terms such as “processing,” “computing,” “calculating,” “determining,” “displaying,” or the like, refer to the action and processes of a computer system, or similar electronic computing device, or data processing device that manipulates and transforms data represented as physical (electronic) quantities within the computer system's registers and memories into other data similarly represented as physical quantities within the computer system memories or registers or other such information storage, transmission or display devices. Computer or electronic operations described in the specification or recited in the appended claims may generally be performed in any order, unless context dictates otherwise. Also, although various operational flow diagrams are presented in a sequence(s), it should be understood that the various operations may be performed in other orders than those which are illustrated or claimed, or operations may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “in response to”, “related to,” “based on”, or other like past-tense adjectives are generally not intended to exclude such variants, unless context dictates otherwise. As used herein the term “simultaneous” is meant to describe processes or events communicated, shown, presented, etc., substantially at the same time. The term “simultaneous” may refer to a level of computer responsiveness that a user perceives as substantially at the same time or that enables the computer to keep up with various external processes at substantially the same time. However, in computer technology, the term “simultaneous” may not refer to events or processes exactly coincident. For example, in signal processing, simultaneous processing may relate to a system in which input data from external components is processed within milliseconds so that it is perceived by the user virtually immediate and simultaneous.

The present disclosure generally relates to medical devices, and it exemplifies embodiments of an optical probe which may be applicable to a spectroscopic apparatus (e.g., an endoscope), an optical coherence tomographic (OCT) apparatus, or a combination of such apparatuses (e.g., a multi-modality optical probe). The embodiments of the optical probe and portions thereof are described in terms of their state in a three-dimensional space. As used herein, the term “position” refers to the location of an object or a portion of an object in a three-dimensional space (e.g., three degrees of translational freedom along Cartesian X, Y, Z coordinates); the term “orientation” refers to the rotational placement of an object or a portion of an object (three degrees of rotational freedom--e.g., roll, pitch, and yaw); the term “posture” refers to the position of an object or a portion of an object in at least one degree of translational freedom and to the orientation of that object or portion of object in at least one degree of rotational freedom (up to six total degrees of freedom); the term “shape” refers to a set of posture, positions, and/or orientations measured along the elongated body of the object.

As it is known in the field of medical devices, the terms “proximal” and “distal” are used with reference to the manipulation of an end of an instrument extending from the user to a surgical or diagnostic site. In this regard, the term “proximal” refers to the portion (e.g., a handle) of the instrument closer to the user, and the term “distal” refers to the portion (tip) of the instrument further away from the user and closer to a surgical or diagnostic site. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute. The term “patient” is generally synonymous with the term “subject” and includes all mammals including humans. Examples of patients include humans, livestock such, and companion animals.

As used herein the term “catheter” generally refers to a flexible and thin tubular instrument made of medical grade material designed to be inserted through a narrow opening into a bodily lumen (e.g., a vessel) to perform a broad range of medical functions. The more specific term “optical catheter” refers to a medical instrument comprising an elongated bundle of one or more flexible light conducting fibers disposed inside a protective sheath made of medical grade material and having an optical imaging function. A particular example of an optical catheter is fiber optic catheter which comprises a sheath, a coil, a protector and an optical probe. In some applications a catheter may include a “guide catheter” which functions similarly to a sheath.

In the present disclosure, the terms “optical fiber”, “fiber optic”, or simply “fiber” refers to an elongated, flexible, light conducting conduit capable of conducting light from one end to another end due to the effect known as total internal reflection. The terms “light guiding component” or “waveguide” may also refer to, or may have the functionality of, an optical fiber. The term “fiber” may refer to one or more light conducting fibers. An optical fiber has a generally transparent, homogenous core, through which the light is guided, and the core is surrounded by a homogenous cladding. The refraction index of the core is larger than the refraction index of the cladding. Depending on design choice some fibers can have multiple claddings surrounding the core.

As discussed in the Background section above, intravascular pressure measurements and intravascular imaging are equally important to diagnose and treat patients suffering of CAD. Decisions on applying the most effective treatment should be taken using both the imaging and fractional flow reserve (FFR) of the vessels. Although pressure measurements should be routine during PCI, in the majority of procedures it remains limited. One of the main reasons for the limited use of pressure measurements during PCI appears to be the increase in cost and time to complete the procedure. Specifically, as outlined above, to accurately measure the intravascular pressure, there is a need for switching between imaging and pressure-sensing catheters. In addition, the need for a pressure wire and a hyperemic drug further increases the risk of side effects due to patient reaction to hyperemic drugs, and may even increase patient discomfort during the procedure. To address these limitations, various embodiments of the present disclosure describe a new hybrid intravascular-imaging and pressure-measuring catheter device. The new catheter device leverages the known optical properties of optical coherence tomography (OCT) catheters, and the mechanical properties of novel membranes which are able to deform in response to very low intravascular pressure changes.

A proposed device is an intravascular OCT imaging catheter having an outer sheath (external sheath), and a smaller internal sheath (inner sheath) sealing an inner imaging core. On the external sheath, a diaphragm-based pressure sensor is arranged over the external sheath distal part. In one embodiment of the pressure sensor, is a cylindrical sensor comprising a circular silicon membrane and a frame placed on the outer surface of the external sheath. An optical fiber of the imaging core, irradiates the silicon membrane with a light beam, and the reflected light is collected by the OCT system producing an image which depicts the diaphragm surface and the inner sheath. The sensitivity of the diaphragm allows small pressure changes (e.g., ±5 mmHg) to deform the membrane, and the OCT images corresponding to the deformed membrane can depict a position of the membrane in a different position than before the pressure change. The difference in distance from the inner sheath to the surface of the membrane is used to calculate the pressure changes between two points of interest, and therefore it is possible to assess the physiological significance of CAD during percutaneous coronary intervention (PCI).

### OCT Intravascular Imaging System

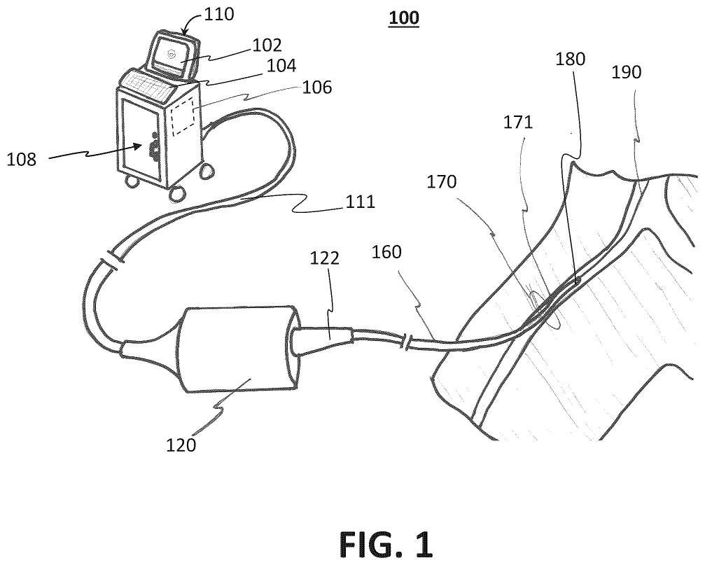

FIG. 1 illustrates a system 100 including a movable cart or console 110, a patient interface unit (PIU) 120, and a multifunction catheter 160. One application for the system 100 described herein is for optical coherence tomography (OCT) imaging of coronary vasculature for diagnosis and/or treatment of coronary diseases and conditions. The system 100 can also be applicable to other catheter-based imaging modalities, such as intravascular ultrasound (IVUS) imaging, near-infrared auto-fluorescence (NIRAF) imaging, near-infrared spectroscopy (NIRS) imaging, and multimodality imaging techniques such as OCT-NIRAF, NIRS-IVUS, and other combinations thereof.

The system 100 generally includes the console 110 connected to the PIU 120 via a cable bundle 111. The PIU 120 is configured to removably connect the catheter 160 to console 110. The PIU 120 and catheter 160 are part of the sample arm for the interfereometer of the OCT system. The console no is configured to control the overall functions of the catheter 160, and to provide a user interface (e.g., a display 102 and keyboard 104) for interaction of the user (a physician) with the system 100. The console 110 may also include a controller or computer 106, and other hardware components 108, such as a source of radiative energy suitable for imaging a biological lumen, and one or more detectors, and other peripherals. In an OCT system for intravascular procedures, the source of radiative energy can include a laser source with a center wavelength of about 1310+/−50 nanometers (nm) that can be used to acquire OCT images of a patient's vessel or other biological lumen. Components 108 may include an interferometer reference arm. In an intravascular ultrasound (IVUS) system, the source of radiative energy can include an ultrasound transducer that emits ultrasound waves in the 10-40 MHz range.

OCT images can be acquired by controlling an imaging core arranged inside the catheter 160. To that end, the PIU 120 may include a fiber optic rotary joint (FORJ) and a pullback unit, which are not shown in the drawings, but are well known components of conventional OCT systems. Examples of PIU including a FORJ are described, for example, in U.S. Pat. Nos. 9,869,828, 10,895,692, and 11,061,218 which are incorporated by reference herein for all purposes

In an intravascular procedure, the catheter 160 is inserted into a blood vessel 170 via an introducer or a guide catheter. Then, the distal end of the catheter 160 is guided to a region of interest (e.g., stenosis) under image guidance. A secondary imaging modality such as an X-ray fluoroscopy system can be used to monitor the insertion of the catheter 160 through the blood vessel 170. When the distal end of the catheter 160 is placed at the site of interest (e.g. a stenosis or stent), the imaging core undergoes a calibration process whereby the imaging core is set to a nominal position (a “home” position) before initiating an imaging operation. In an imaging operation, the catheter 160 can scan a vessel wall 171 with a beam of radiative energy from the laser source to acquire one or more images. As used herein the term “scan” or “scanning” refers to the process, act, or instance of scanning (as by passing a beam of radiative energy over or through) an object in order to obtain imaging data representative of the morphologic structure and/or chemical composition the object.

The system 100 shown in FIG. 1 is similar to conventional OCT systems currently known in the field of intravascular imaging. There are numerous patent publications and non-patent publications that describe conventional imaging catheters which may also include means for determining fractional flow reserve (FFR) based on measurements of intravascular pressure. See, for example, U.S. Pat. Nos. 10,307,070B2, 8,715,200 B2, US 2015/0367105 A1, U.S. Pat. Nos. 8,961,452 B2, 5,902,248, 10,130,269 B2, and 10,932,670B2, all of which are described in the Background section of the present disclosure, and are incorporated by reference herein for all purposes. The OCT catheter system 100 of the present application includes a hybrid catheter equipped with an imaging core configured to acquired OCT images, and a novel pressure sensor 180 configured to measure intravascular pressure inside coronary arteries and/or circulatory veins.

### Hybrid Catheter Configured to Acquire Pressure Data and Image Data from a Vessel

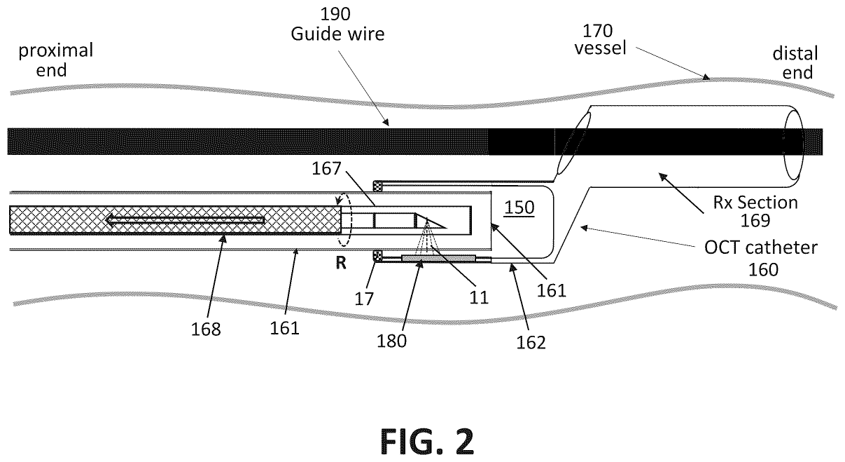

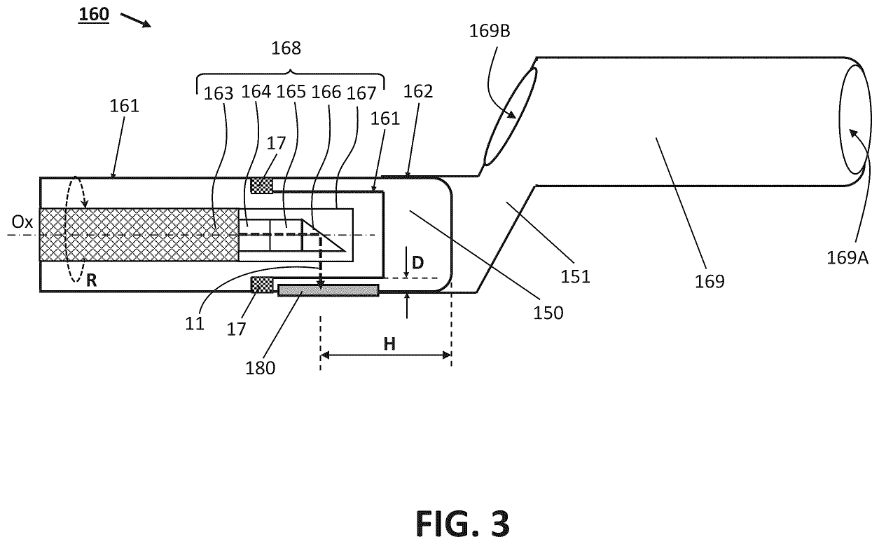

A detailed structure of the catheter 160 comprising a pressure sensor 180 is described with reference to FIG. 2 and FIG. 3. FIG. 2 illustrates a first embodiment of the catheter 160 inserted into a vessel 170. The catheter 160 is inserted into the vessel 170 (e.g., a coronary artery) by guiding the distal end of the catheter over a guidewire 190. According to one embodiment, the catheter 160 includes an imaging core 168 provided inside an inner sheath 161; and the inner sheath is arranged at least partially inside an outer sheath 162. In at least some embodiments, the outer sheath 162, the inner sheath 161, and the imaging core 168 are centered on a longitudinal axis Ox, so as to be coaxial to each other. The imaging core 168 includes of a wire torque coil 163 (torque transferring component), an optical fiber 164 and a distal optics assembly enclosed by a transparent window 167. The outer sheath 162 surrounds at least part of the inner sheath 161 and at least part of the imaging core 168. In other words, the inner sheath 161 is nested inside at least part of the outer sheath 162, and the imaging core 168 is arranged inside the inner sheath 161. The inner sheath 161 and the outer sheath 162 are attached to each other by mechanical attaching means 17, so as to create an empty space or chamber 150 around the distal end of the inner sheath 161.

A diaphragm-based pressure sensor 180 is arranged on the outer sheath 162 at or near the distal end of the inner sheath 161. The pressure sensor 180 is integrally formed with the outer sheath 162, or is attached to the outer sheath 162 by mechanical means such as by bonding with adhesive material, or by welding. More specifically, the pressure sensor 180 is arranged on the outer sheath such that the most distal portion of the imaging core 168 (e.g., the transparent window 167 of the imaging core) is aligned with the pressure sensor 180. When the catheter 160 is inserted into the vessel 170, any fluids contained within the vessel 170 completely surround the catheter, and therefore the pressure sensor 180 can detect intravascular pressure. To detect pressure, the optical fiber scans a diaphragm membrane and the inner sheath with a light beam 11. Light reflected or scattered by the membrane and the inner sheath is collected by the OCT system producing an image which depicts the diaphragm surface and the inner sheath. When the optical fiber is pullback, the OCT system acquires images of the vessel.

FIG. 3 shows an embodiment of the catheter 160 without the vessel 170. In this embodiment, the distal end of the catheter includes a rapid exchange section (Rx section 169). A slanted portion 151 joins the outer sheath 162 to the Rx section 169. The slanted portion 151 maintains the lumen of the outer sheath 162 hermetically sealed, and also keeps the Rx section 169 offset with respect to the longitudinal axis Ox of the catheter. The Rx section 169 includes an entry port 169A and an exit port 169B with a guidewire lumen extending therethrough. The guidewire lumen has a diameter dimensioned to pass therethrough the guidewire 190. In one embodiment, the guidewire 190 can have a diameter in a range of 0.014 to 0.038 inches, and is used to navigate the tip of the catheter 160 to a region of interest (e.g., a stenosis) within the vessel 170. In some embodiments, the Rx section 169 may be an optional feature of the catheter 160.

In at least some embodiments, the outer sheath 162 comprising the sensor 180, and the Rx section 169 can be fabricated as a separate component configured to be mounted onto the inner sheath 161. For example, in one embodiment, the outer sheath 162 is coupled to the inner sheath 161 to overlap only a distal portion thereof. In this embodiment, the length of the inner sheath 161 is different from the length of the outer sheath 162; and a proximal portion of the outer sheath 162 is attached to the distal portion of the inner sheath 161, by mechanical attaching means 17 such as pressure fitting, welding or gluing. In alternative embodiments, the length of the inner sheath 161 can be substantially the same as the length of the outer sheath 162, and the outer sheath 162 comprising the pressure sensor 180 can be coupled to the inner sheath 161 at the proximal end and at the distal end, so that the entire length of the catheter 160 can have a double sheath structure. In some embodiments, as long as the inner sheath 161 is at least partially inserted into the outer sheath 162, the two sheaths do not need to be coupled to each other as long as both sheaths can remain stationary when the imaging core is rotated and/or pullback. In some embodiments, the outer sheath 162 may not include the slanted portion 151 that connects to the Rx section, so that the catheter 160 can be introduced into the vessel without the use of a guidewire.

As it will be appreciated by persons having ordinary skill in the art, when the inner sheath and the outer sheath are manufactured as separate components, in the event that the pressure sensor 180 fails (e.g., when the membrane brakes), the outer sheath 162 and sensor 180 could be removed and replaced with a new one. Alternatively, the outer sheath 162 and sensor 180 could be removed, and the catheter could continue to be used for imaging purposes only, without using the pressure sensor. As a further alternative, in the event that the pressure sensor 180 fails (e.g., the membrane brakes or punctures), the outer sheath 162 attached to the inner sheath, but nevertheless the catheter can continue to be used for imaging purposes without measuring intravascular pressure.

The imaging core 168 is arranged inside the inner sheath 161 substantially coaxial with an inner lumen thereof. The lumen of the inner sheath 161 is hermetically sealed so as to prevent any fluids from entering the imaging core 168. The inner sheath 161 can be dimensioned to receive the imaging core 168 within its lumen with a minimum tolerance between the maximum diameter of the imaging core and the inner diameter of the inner sheath 161. One or more of a lubricious material and a centering tube can be used within the inner sheath so that the imaging core 168 rotates freely about catheter axis Ox while the inner sheath 161 and the outer sheath 162 remain stationary during pullback. Here, it is noted that when the inner sheath 161 and the outer sheath 162 remain stationary during a pullback procedure, the imaging core guides a light beam 11 to the membrane 182, by transmitting the light beam through the inner sheath 161 and through the chamber 150. At least a portion of the light is reflected or scattered by the membrane 182 and by the inner sheath 161. When the inner sheath 161 reflects light, the reflected light will provide a very stable signal which is used as a fixed reference to calculate a distance (an amount of bending) of the pressure membrane 182. Therefore, an advantage of the hybrid catheter 160 is that the inner sheath provides a stable reference for distance measurement (deflection measurement) which produces more accurate intravascular pressure results, and if the sensor membrane breaks the catheter can still be used for imaging because the imaging core is hermetically sealed inside the inner sheath 161.

The imaging core 168 is comprised of the torque transfer component (e.g., a torque coil 163), a waveguide component (e.g., a double clad optical fiber) 164, and an assembly of distal optics. The distal optics may include at least a focusing component (e.g., a GRIN lens or ball lens) 165 and a beam directing component (e.g., a mirror or total internal reflection surface) 166. A transparent window 167 encloses the distal optics at the distal end of the torque coil 163. In some embodiments, the focusing component and the beam directing component can be formed by a single component, for example, by a polished ball lens formed at the distal end of the optical fiber. In an OCT modality, a light beam 11 is transmitted from a light source (not shown) to the distal optics assembly via the PIU 120. The light beam 11 is guided by the beam directing component 166 such that the light beam exits through the inner sheath 161 at an angle with respect to the catheter axis Ox. The pressure sensor 180 is arranged in the wall of the outer sheath 162 at a distance H from the distal end thereof. Distance H can correspond, for example, to the nominal or “home” position of the imaging core 168 (e.g., the most distal position from which a pullback process starts). The location of the pressure sensor 180 is such that the light beam 11 emitted by the imaging core travels through the inner sheath 161 and irradiates at least a portion (e.g., the center) of a membrane 182 when the imaging core 168 is at its most distal position. In addition, the inner sheath 161 is dimensioned with an outer diameter (OD) which is smaller than an inner diameter (ID) of the outer sheath 162, so that the inner sheath 161 can fit into the lumen of the outer sheath 162 within a certain distance D therebetween. In other words, at least at the location where the pressure sensor 180 is attached to the outer sheath, there is a void or empty space (a chamber 150) between the inner sheath 161 and the outer sheath 162.

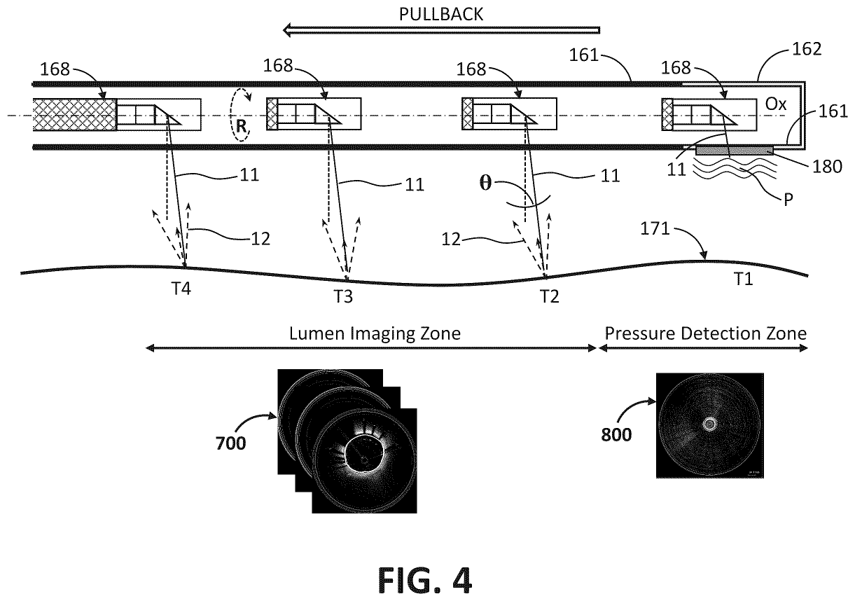

The chamber 150 between the inner sheath 161 and the outer sheath 162 provides space for the membrane 182 to freely bend is response to intravascular pressure of fluids surrounding the catheter tip. In some embodiments, the chamber 150 can be formed by an annular region surrounding the inner sheath in correspondence with the pressure membrane. That is the chamber 150 is an annular space defined by an annular region of the inner sheath that is surrounded by the outer sheath and the membrane 182. In other embodiments, the chamber 150 is defined by a space contained between a distal portion of the inner sheath 161 that is surrounded by the outer sheath 162. That is, the chamber 150 is defined by a cylindrical space that surrounds a distal portion of the inner sheath 161, as shown in FIG. 2 and FIG. 3. In other embodiments, the chamber 150 may extend from the proximal end distally to surround the tip of the inner sheath 161, e.g., as shown in FIG. 4.

In operation, the distal end of the imaging core 168 emits a light beam 11 that travels through the inner sheath 161, through the chamber 150, and then is incident on the sensor 180. The inner sheath 161 and the pressure sensor 180 both cause at least a portion of the light beam to be reflected or backscattered, and this light is collected by the distal optics assembly of the imaging core. When the catheter 160 is surrounded by intravascular fluids (e.g., blood or flushing agent), the chamber 150 has no fluid communication with the fluids in the vessel. Therefore, even a small amount of pressure P causes a thin membrane 182 of the pressure sensor 180 to deform or deflect towards the inner sheath 161. This change in shape (or deformation) of the pressure membrane 182 modulates the light of the light beam 11. Therefore, when the backscattered light is detected and processed by the OCT system 100, a computer or processor of the system can calculate the intravascular pressure P based on the amount of deflection, as further described below.

FIG. 4 illustrates an exemplary longitudinal view of catheter 160 undergoing a data collection process, according to the present disclosure. As understood from FIG. 1, catheter 160 is connected at the proximal end thereof to the PIU 120 via a catheter connector 122. When the catheter 160 is connected to the PIU 120, the torque coil 163 delivers torque (rotational force) generated by a non-illustrated rotational motor located inside the PIU 120. In addition, a pullback unit (e.g., a translation stage) moves the torque coil 163 in a linear direction, while the inner sheath 161, the outer sheath 162, and the pressure sensor 180 remain stationary. At the distal end of the catheter 160, the beam directing component (e.g., a mirror, a prism, or a grating) guides the light beam 11 sideways toward the vessel wall 171. According to the present disclosure, the pressure sensor 180 is arranged on the surface of outer sheath 162 at a predetermined distance from the inner sheath 161. Since the imaging core 168 is configured for side-view imaging, where the light beam 11 is incident on the vessel wall 171, the light beam 11 is directed at a small angle theta (θ) with respect the normal to the catheter axis Ox.

At the beginning of an intravascular procedure, the distal tip of catheter 160 is navigated to a region of interest (e.g., stenosis). Before initiating a pullback operation (e.g., before clearance of the fluids that surround the catheter tip), the light beam 11 is incident on the pressure sensor 180. As mentioned above, the fluids surrounding the distal tip of the catheter 160 causes a membrane 182 of the pressure sensor 180 to bend or change its shape (to deform or deflect from its initial position). Here, since OCT data and pressure data are obtained by the same imaging core 168, the light beam 11 emitted from the imaging core 168 first irradiates the pressure sensor 180, and subsequently irradiates the vessel wall 171 (tissue) of vessel 170, in a single pullback procedure (a single pass).

During a pullback operation shown in FIG. 4, the imaging core 168 can be controlled to first align the light beam 11 with the pressure sensor 180, and scan the membrane 182 through the inner sheath 161, before initiating pullback. More specifically, when the catheter 160 is inserted into the vessel 170, the catheter 160 can be momentarily stopped (parked) at a pressure detection zone distal to a region of interest (i.e., distal to a stenosis). In the pressure detection zone, the system controls the imaging core 168 to scan the membrane 182 by transmitting the light beam through the inner sheath 161 until intravascular pressure is established. The imaging core 168 scans the inner sheath 161 and the membrane 182 while rotating or oscillating the light beam 11, but without being pullback. In some embodiments, the imaging core 168 can be configured to scan the pressure sensor while rotating around the catheter axis, and translating only a small distance (e.g., a distance equal to the radius of the membrane 182). In any case, light reflected or scattered by the pressure sensor 180 carries information about the deformation of the membrane 182, and light reflected or scattered by the inner sheath 161 serves as a steady (fixed) reference for determining an amount of deflection of the flexible membrane 182. Here, the system generates at least one OCT image 800 in which the pressure membrane 182 and the inner sheath 161 are imaged simultaneously. Then, after intravascular pressure is measured, blood clearance can be trigged, and pullback is started.

After pullback is started, while the light beam 11 scans the tissue of vessel 170 (e.g., an artery wall), the imaging core 168 rotates or oscillates inside the inner sheath 161 (as indicated by arrow R) around the catheter axis Ox. At the same time, the imaging core 168 is pulled back (translated from the distal end to the proximal end of the stenosis), while the inner sheath 161, outer sheath 162 and pressure sensor 180 remain stationary. The imaging core 168 collects returning light 12 which includes light reflected and/or scattered by the vessel wall 171. In this process, the system generates a series of OCT images 700 of the vessel wall 171 based upon the light reflected and/or scattered by the vessel wall 171.

In this manner, a first interference signal can be obtained by combining a reference light beam (not shown) and the collected light reflected or scattered by the pressure sensor 180 and by the inner sheath 161, and a second interference signal can be obtained by combining the reference light beam with the collected light reflected or scattered by the vessel wall 171. The catheter 160 is configured to sequentially scan the pressure sensor 180 at least at a first position T1, and the vessel wall 171 at a plurality of positions T2, T3, T4, etc. Here, for example, at each position T1-T4, the imaging core 168 may complete at least one full rotation in advancing from one position to the next. As explained above with reference to FIG. 1, the interference OCT signal is converted into an electrical signal, which is the digitized, stored, and/or processed by one or more processor of computer 106 to generate at least one OCT image 800 of the pressure sensor, and a plurality of OCT images 700 of the vessel wall 171.

The combination of backscattered light (returned light 12) and reference light from the reference beam (not shown) results in the OCT interference signal, only if light from both the sample and reference beams have traveled substantially the same optical distance. As used herein, “substantially the same optical distance” indicates a difference of less than or equal to the coherence length of the light source. Regions of the vessel 170 that reflect more light will create stronger interference signals than regions that reflect less light. Any light that is outside the coherence length will not contribute to the interference signal. The intensity profile of the reflected light, which also referred to as an A-scan or an A-scan line, generally contains information about the spatial dimensions and/or location of characteristic features within the vessel 170. In the present disclosure, the OCT signal also includes information of the pressure experienced by the pressure sensor 180. An OCT image (i.e., a cross-sectional tomograph generally referred to as a B-scan) may be formed by combining multiple adjacent A-scans at different positions along the lumen.

The diagram of FIG. 4 depicts the imaging core 168 scanning the pressure sensor 180 and the vessel wall 171 at a plurality of positions T1, T2, T3, T4, etc., in the lengthwise direction of the catheter 160 along the pullback path. During a single pullback, the system collects OCT interference signals, while the imaging core scans first the pressure sensor 180, and then the vessel wall 171 with the illumination light beam 11. In this manner, the catheter tip can have a first scanning range for “Pressure Detection” in which the OCT signals corresponding to pressure measurement are collected, and a second scanning range for “Lumen Imaging” where the OCT signals correspond to imaging of the vessel wall 171 are collected. More specifically, at the beginning of the pullback operation, i.e., at a first longitudinal position T1, the light beam 11 first scans only the pressure sensor 180. Then, at subsequent positions, T2, T3, T4, etc., the light beam 11 scans the vessel wall 171 (inner surface of a bodily lumen).

Measurements at each location are performed while continuously rotating the imaging core 168 and irradiating first the pressure sensor 180, and subsequently the vessel wall 171 with light beam 11 at a fixed angle θ. Naturally, as a matter of course, the system can also collect pressure measurements after the pullback operation (i.e., at the proximal side of the stenosis).

The linear pullback movement combined with rotational movement R of the catheter 160 enables A-lines to be generated multiple times by helically scanning first the pressure sensor 180, and subsequently the vessel wall 171. Combining the plurality of A-line scans allows the generating of a 2D image or B-scan. Each 2D image of an artery cross section, for example, may be formed by approximately 500 lines or more, corresponding to at least one full circumferential scan (360 degree scan) by the catheter 160. This full circumferential scan is also sometimes referred to as a “frame”. Three-dimensional (3D) imaging of the vessel wall 171 can be achieved by combining plural 2D image frames obtained during the longitudinal translational motion of the pullback operation while the imaging core is rotated. The resulting catheter scan is a helical path of successive A-lines to form a full 3D dataset of the vessel wall 171, as it is well known in the art. The same type of scanning operation can be performed at the distal and proximal ends of a stenosis to scan the pressure sensor 180, and acquire intravascular pressure with the same catheter. Each 360-degree rotation (full revolution) scan within the helical path may also be referred to as a frame, and multiple frames can be generated along the longitudinal (z axis) direction in the minus z-direction. Data collected from successive A-line scans is processed (e.g., by fast Fourier transformation and other known algorithms) to generate OCT images of the vessel 170 in a known manner. At the same time, the OCT signal from the pressure sensor 180 is also collected, pressure is calculated based on the OCT signal, and intravascular pressure results are displayed and analyzed in correspondence with the OCT images of the vessel 170.

### Exemplary Embodiment of Pressure Sensor 180

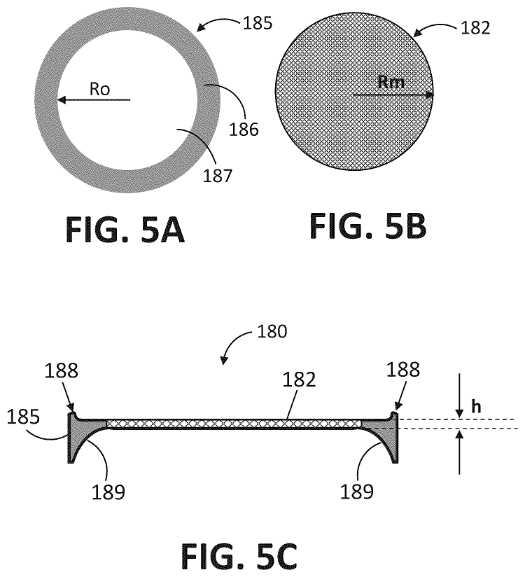

FIG. 5A, FIG. 5B, and FIG. 5C illustrate a first embodiment of the pressure sensor 180 useful for monitoring blood pressure and/or FFR in a vessel. According to the present disclosure (including the claims), an element such as a detector, which may take the form of a sensor, that is “useful for monitoring” pressure or FFR needs only play a role in the monitoring, and needs not completely perform all the steps necessary to achieve the monitoring. Also, in present disclosure (including the claims), monitoring pressure and/or FFR in a vessel “using” a sensor or a detector needs that the sensor or detector play only a certain role (be involved), in the monitoring, but needs not be the sole component used to achieve the monitoring. As used herein, the term “pressure” is defined as the force or strain applied by a fluid (liquid or gas) on a surface; and this force or strain can be measured in units of force per unit of surface area. Common pressure units are Pascal (Pa), Bar (bar), N/mm² (Newton pre millimeter square) or psi (pounds per square inch). However, blood pressure is commonly given in mean values of millimeter of Mercury (mmHg), where 1.0 mmHg is equivalent to 133.322387415 Pa or 0.01933678 psi. The term “sensor” is defined as a device that measures a physical quantity and translates it to a signal. A pressure sensor is an instrument consisting of a pressure sensitive element to determine the actual pressure applied to the sensor (using different working principles) and some components to convert this information into an output signal. In the present disclosure, the physical quantity being measured is pressure (or more specifically “fluid pressure” within a bodily lumen), and the output signal is in most cases electrical. The electrical signal is the digitized, stored, and/or processed by one or more processors of computer 106 to calculate an amount of intravascular pressure. The intravascular pressure results are displayed and analyzed in correspondence with the OCT images of the vessel 170.

According to one embodiment, the pressure sensor 180 is constructed as a diaphragm made of an ultrathin silicon membrane 182 attached to a frame 185. The silicon membrane 182 can have a thickness “h” of about 200 nm. The thickness “h” can range from about 100 nm to 1000 nm or from about 100 nm to 500 nm to make the diaphragm membrane 182 more or less sensitive to pressure changes. The pressure sensor 180 is configured to be positioned in the optical path of (e.g., perpendicular to) the light beam 11. For example, FIG. 2 shows an embodiment where the pressure sensor 180 is arranged substantially parallel to the catheter axis Ox, and a light beam 11 in incident on the pressure sensor at various angles. As along as the pressure sensor 180 intersects the optical path of light beam 11 such that sufficient light is reflected or backscattered by the sensor membrane, and collected by the distal optics assembly, the angle of light beam 11 is not limited.

FIG. 5A shows a top view of frame 185; and FIG. 5B shows at top view diaphragm membrane 182, according to one embodiment. FIG. 5C illustrates a side-view of the diaphragm membrane 182 assembled with the frame 185. As shown in these drawings, the diaphragm membrane 182 can be a thin circular membrane having a radius r and a thickness h. In one example, the membrane 182 is a circular silicon membrane. The material for the flexible membrane 182 is not limited to silicone. In general, the membrane can be made from any elastic material (e.g., cross-linked polymer with reflective or fluorescent particles) that is resilient and can recover from deflection induced by intravascular pressure.

The frame 185 has a cylindrical shape, comprising a cylindrical edge 188, a substantially flat surface 186 with an opening 187, and a bottom arcuate surface 189. The opening 187 has a radius Ro slightly smaller than a radius r of the diaphragm membrane 182 (i.e., r>Ro). An adhesive (e.g., glue) or welding can be used on the flat surface 186 to attach the flexible membrane 182 to frame 185. The arcuate surface 189 is configured to sit on the cylindrical surface of the outer sheath 162. To ensure sufficient space for the diaphragm membrane 182 to deform under the fluid pressure, there is a distance D between the surface of membrane 182 and the outer surface of inner sheath 161. In other words, at least at the location where the pressure sensor membrane is attached to the catheter outer sheath, there is an empty space or chamber 150 between inner sheath 161 and the outer sheath 162, as shown in FIG. 3. In addition, the outer sheath 162 has a side opening that coincides with the opening 187 of the frame 185. Since the bottom arcuate surface 189 of frame 185 is configured to adapt to the cylindrical surface of outer sheath 162, the outer diameter of catheter 160 can be maintained to a minimum while still providing enough space (distance D) between the inner sheath 161 and the outer sheath 162 for the diaphragm membrane 182 to become deformed in response to intravascular pressure. When the pressure sensor 180 is mounted onto the outer sheath 162, the pressure membrane 182 is substantially tangential to the outer surface of outer sheath 162 and parallel to the catheter axis Ox. The frame 185 can be a piece of medical-grade biocompatible metal or biocompatible plastic material shaped into a cylinder (e.g., a laser-cut, molded or 3D printed tube), and provided with a substantially flat surface 186 with an opening 187. A person of ordinary skill in the art will appreciate that flexible membrane 182 and frame 185 are not limited to cylindrical and circular shapes. The membrane 182 can be of a polygonal membrane (e.g., a rectangular membrane), and the frame 185 can be a polygonal cylinder (e.g., a rectangular cylinder). Naturally, however, when the membrane 182 is made of other shapes and materials, calculations for the membrane deflection will have to be modified accordingly based on the teachings disclosed herein.

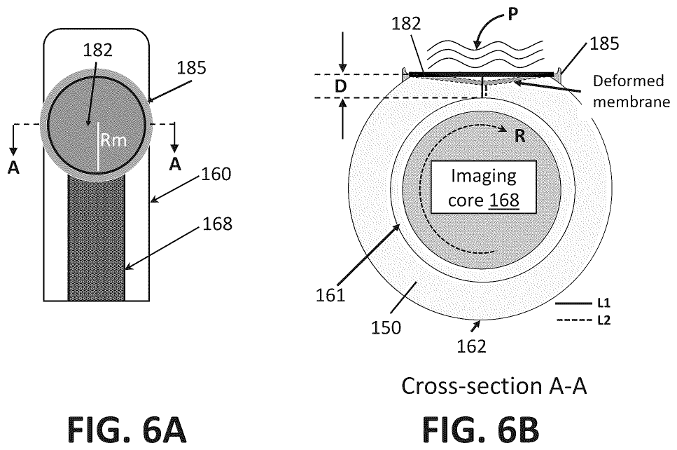

FIG. 6A and FIG. 6B show a more detailed view of the pressure sensor 180 mounted on the outer sheath 162. FIG. 6A shows a top view along the longitudinal direction of the catheter (a view in the lengthwise direction parallel to the catheter axis Ox). As seen in FIG. 6A, the sensor 180 is attached to the catheter 160 near the distal end thereof. FIG. 6B shows a cross-sectional view taken across section A-A (a view perpendicular to the catheter axis Ox) of the pressure sensor 180 attached to the outer sheath 162. As shown in FIG. 6A, the diaphragm frame 185 holds the diaphragm membrane 182 attached to the outer sheath 162 near the distal end thereof. In this embodiment, the frame 185 has a cylindrical edge 188. The cylindrical edge 188 protrudes slightly above the membrane 182 to protect the membrane 182 from being scratched or punctured. The diameter of cylindrical edge 188 can be equal to or smaller than the diameter of outer sheath 162. The frame 185 can be made by molding (or 3D printing) biocompatible plastic materials (e.g., Polyvinylchloride, Polyethersulfone, Polyethylene, or other similar materials) into an arcuate shape that fits on the outer surface of outer sheath 162. Then, the frame 185 is permanently attached (bonded or laser welded) to the outer surface of the outer sheath 162 at a predetermined position (a pressure detection position) at the distal end of the catheter 160. FIG. 6B shows the working principle of the pressure sensor 180.

### Methods of Acquiring Pressure Data and Image Data from a Bodily Lumen Using a Single Pullback of an OCT Catheter Having a Pressure Sensor

As shown in FIG. 6B, the fluid surrounding the distal tip of catheter 160 will exert a pressure P on the diaphragm membrane 182, and this pressure P will cause the membrane 182 to deform or change its shape from a substantially flat surface to a curved or bent surface. Initially, when the catheter is not surrounded by fluid, the diaphragm membrane 182 is a first distance Li from the inner sheath 161. Then, when the catheter is inserted in a bodily lumen (e.g., a blood vessel), pressure from the fluids contained in the bodily lumen will surround the catheter tip and cause the diaphragm membrane 182 to bend and move towards the inner sheath 161. Under pressure P, the membrane 182 will be at a second distance L2 (where L2*L1) with respect to the inner sheath 161. The change in distance between the flexible membrane 182 and the inner sheath 161 is proportional to intravascular pressure (in particular blood pressure).

To calculate the pressure (P) from the membrane's deformation, known mathematical theory can be used. According to one embodiment, we use the DiGiovanni elasticity equation described by M. Di Giovanni, in “Flat and Corrugated Diaphragm Design Handbook”, published by Routledge, 2017.

According to DiGiovanni, the pressure (P) is given by Equation (1)

$$P = [ \frac{16}{3 ( 1 - {μ}^{2} )} ( \frac{E {h}^{3}}{{r}^{4}} ) ] d + [ \frac{( 7 - μ )}{3 ( 1 - μ )} ( \frac{E h}{{r}^{4}} ) ] {d}^{3} , \qquad Eq . ( 1 )$$

where E is the Young's modulus, μ the Poisson's ratio, r the radius, h the thickness, and d is the deflection (d=L1−L2) of the membrane 182 caused by the pressure P.

The change in distance or deflection “d” can be compared to calibration data for the membrane, which can be previously stored as part of the system settings. The calibration data can include tabulated values of the relationship of deflection distance d to pressure P of the membrane, which can be obtained experimentally based on the Young's modulus E, the Poisson's ratio μ, the radius r, and the thickness h of membrane 182.

In other embodiments, the deflection (d=L1−L2) of the membrane 182 caused by the pressure P can be calculated using other techniques. For example, the non-bent position of the membrane 182 can be designed to be tangent to the outer surface of outer sheath 162 (see FIG. 6B). Since the outer diameter of the outer sheath 162 and the outer diameter of the inner sheath 161 are known parameters of the system (i.e., the distance D in FIG. 6B is a predetermined distance), the bent position of the membrane 182 can be determined from the OCT image acquired by the imaging core during pressure detection. Then, the system 10 can calculate intravascular pressure using a deflection-to-pressure relationship similar to that disclosed by U.S. Pat. No. 10,932,670. Advantageously, the present disclosure uses the inner sheath 161, which is stationary relative to the rotating imaging core, as a fixed and reliable reference to more accurately measure the deflection of membrane 182.

Other methods for calculating the amount of deflection of membrane 182 can be based on analyses of the OCT image of membrane acquired by the OCT system. For example, a B-scan image acquired by the OCT catheter 160 during pressure detection and displayed in in Cartesian coordinates will show light reflected or scattered by the inner sheath 161 and by the membrane 182 as well defined circles. Therefore, in a B-scan image acquired during pressure detection, the distance between pressure membrane 182 and inner sheath 161 can be calculated by analyzing the peak signals corresponding to each circle. To obtain a more accurate measurement, an average of a plurality of B-scan images can be analyzed. Similar analysis can be done in B-scan images represented in polar coordinates.

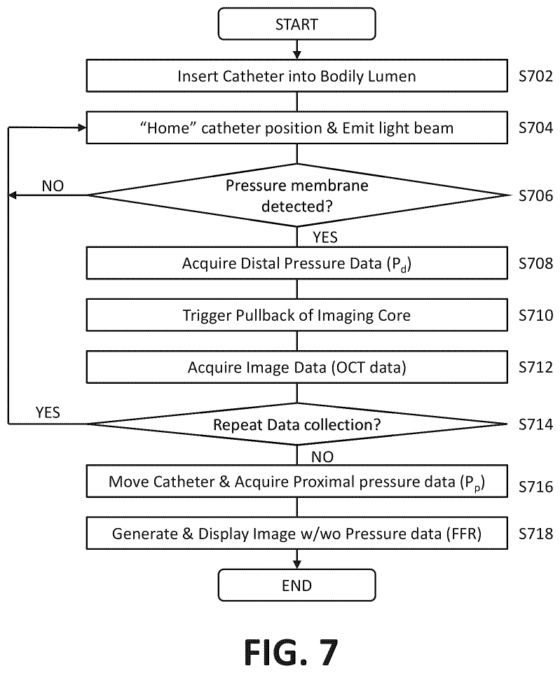

FIG. 7 is flowchart illustrating an exemplary process (method) of acquiring intravascular pressure and image data using a single pullback of the multifunctional catheter 160. The workflow starts at step S702, when the catheter 160 is navigated to a region of interest (e.g., stenosis) inside a bodily lumen, such as a vessel 170. At step S704, the catheter 160 is calibrated and positioned to a “home” position. At this point, the imaging core starts emitting a light beam 11. At step S706, the processor determines if the detector receives light reflected and/or scattered from the diaphragm membrane 182. Here, if S706=NO, the calibration and/or homing procedure of the catheter may be repeated. To improve alignment of the light beam with the membrane 182, the membrane can be made of specific materials configured to reflect or scatter light of the light beam 11. Alternatively, or additionally, at least the distal portion of inner sheath 161 (i.e., the portion concentric with the pressure membrane 182) could be made of glass doped with scattering agents such as Titanium oxide (TiO₂) or Barium sulfate (BaSo₄). In some embodiments, the material of the inner sheath 161 may include well known polymers such as FEP, PET, PTFE, nylon and/or combinations thereof with or without doping (undoped) enhance alignment with the membrane 182. FEP (fluorinated ethylene propylene), PTFE (polytetrafluoroethylene: a synthetic fluoropolymer of tetrafluoroethylene) and FEP (polyethylene terephthalate) are similar in their material properties, and are widely used in medical devices due to their biomedical compatible properties. Any of the forgoing implementations, materials, or combinations thereof can be used to ensure the inner sheath 161 provides a clear and steady reference signal to more accurately calculate the amount of deflection d of the membrane 182.

In this manner, step S706 ensures that light beam 11 is properly aligned with the membrane 182 of pressure sensor 180. If the processor determines that the pressure membrane is detected (S706=YES), the workflow advances to step S708. At step S708, the processor acquires distal pressure data (P_{d}). As explained above, for FFR calculation, pressure data distal to a stenosis (P_{d}) and pressure data proximal to stenosis (Pₚ) is necessary. Here, pressure data can be acquired by detecting light reflected or scattered by the membrane 182 and by the inner sheath 161 while the imaging core is rotating, or even without rotating the imaging core. For example, once the processor determines that the pressure membrane is aligned with the light beam 11, rotation of the imaging core can be stopped, and the light reflected or scattered by the pressure membrane 182 can be collected for a predetermined amount of time. Alternatively, and more preferably, light reflected or scattered by the inner sheath 161 and by the pressure membrane 182 can be simultaneously collected for at least one full revolution of the imaging core 168. For more accurate measurement of the intravascular pressure, light reflected or scattered by the inner sheath 161 and by the pressure membrane 182 can be collected for several revolutions of the imaging core 168, so that the OCT system can generate clear image OCT 800. Therefore, acquiring pressure data refers to detecting light reflected or scattered by the flexible membrane 182 and by the inner sheath 161, calculating an amount of deflection of membrane 182, and calculating the distal pressure (P_{d}). In one embodiment, the distal pressure can be calculated according to DiGiovanni pressure equation given by Equation (1), where the amount of deflection (d) of the membrane 182 can be obtained from the OCT image 800 of the membrane.

At step S710, after the pressure data distal to a stenosis (P_{d}) is determined, a pullback process can be triggered. In at least one embodiment, the actual trigger of pullback and recording of OCT images can be based on the positive detection of intravascular pressure distal to a stenosis (P_{d}). Positive detection of distal pressure (P_{d}) can be based on certain known thresholds, including, for example, the mean arterial pressure. After the distal pressure is detected, blood clearance (flushing) can be started. Then pullback can be triggered shortly after blood clearance.

After the pullback is triggered, the imaging core scans the bodily lumen (e.g., the vessel wall 171) as illustrated in the “lumen imaging” example shown in FIG. 4. At step S712, processor collects image data (e.g., OCT data) for the amount of the pullback length (e.g., 50 mm or 80 mm). Acquiring OCT data includes collecting light backscattered from the vessel wall 171, interfering the collected light with light of a sample arm of the interferometer, and detecting and processing the interference signals. At step S714, the system may prompt the user as to whether to repeat the data collection process. If S714=YES, the workflow returns step S704. If S714=NO, the process advances to step S716. At step S716, the user may partially withdraw the catheter to place the catheter tip proximal to the region of interest (e.g., proximal to the stenosis). When the catheter tip is proximal to the region of interest, the user may again acquire pressure data in a manner similar to steps S706-S708. Here at step S716, catheter 160 is used to acquire pressure data proximal to stenosis (Pₚ) by again detecting light reflected or scattered by the membrane 182 and the inner sheath 161. In this manner, intravascular pressure can be calculated before and after the pullback.

At step S718, the system generates one or more images from the image data, and displays the one or more images along with or without the pressure data (e.g., FFR). Here, OCT images can be generated as a collection of cross-sectional images, which can be displayed as individual images or as a video sequence. Scrolling through adjacent cross-sectional images can enable an experienced physician to obtain a three-dimensional assessment of the vessel segment. Automated image reconstruction techniques can also generate a wireframe image, which can give the operator a more natural 3D view of the entire vessel segment.

Given that pressure data and image data are acquired by the same imaging core in a single pullback, pressure data can be shown in relation to the stenosis on the OCT longitudinal mode or L-mode. If OCT data is used to generate a wireframe model of the vessel, the diameter and length of the vessel segment can be used to display the stenosis on the L-mode. In addition, given a wireframe model of a stent, visual guidance for stent placement can be displayed. Regardless of how the OCT images and pressure data are presented, the simultaneously acquired pressure data and image data of a vessel can be used during pre-stenting, stenting, and post-stenting stages of an intravascular procedure.

As mentioned above, decisions on diagnosing CAD and applying the most effective PCI treatment should be made using both vessel imaging and fractional flow reserve (FFR) or other such pressure measurement parameter. FFR derives from intracoronary pressure measurements along at least two locations of the vessel; FFR is the ratio of the distal to a stenosis pressure (P_{d}) to the proximal to stenosis pressure (Pₚ), and is the current ground truth for PCI. Advantageously, according to the present disclosure, calculating the FFR based on the membrane displacements as shown in FIG. 6B can reduce the time and cost of the procedure because pressure data and image data can be acquired by a single scanning pass of the same catheter. The use of coaxially nested catheter sheaths (i.e., the inner sheath 161 nested inside the outer sheath 162) enables accurate calculation of the intravascular pressure difference between distal to stenosis pressure (P_{d}) and proximal to stenosis pressure (Pₚ) because the distance between the two sheaths is known and remains fixed. The pressure is accurately measured by using the light intensity of the light beam 11 reflected or scattered by the membrane 182.

In the event that the diaphragm membrane 182 malfunctions (breaks or is punctured), the outer sheath 162 could be replaced for a new one. Alternatively, the catheter can be continued to be used for OCT imaging even when the membrane fails (breaks) because the inner sheath 161 maintains the imaging core 168 sealed, and prevents any fluids from entering into the imaging core.

### Software Related Disclosure

At least certain aspects of the exemplary embodiments described herein can be realized by computer 106 of system 100 that reads out and executes computer executable instructions (e.g., one or more programs or executable code) recorded on a storage medium (which may also be referred to as a ‘non-transitory computer-readable storage medium’) to perform functions of one or more block diagrams or flowchart diagrams described above. The computer 106 may include various components known to a person having ordinary skill in the art. For example, the computer may include signal processor implemented by one or more circuits (e.g., a field programmable gate array (FPGA) or an application specific integrated circuit (ASIC)) for performing the functions of one or more of the above-described embodiment(s), by, for example, reading out and executing the computer executable instructions from the storage medium to perform the functions of one or more of the above-described embodiment(s) and/or controlling the one or more circuits to perform the functions of one or more of the above-described embodiment(s). The computer 106 may comprise one or more processors (e.g., central processing unit (CPU), micro processing unit (MPU)), and may include a network of separate computers or separate processors to read out and execute the computer executable instructions. The computer executable instructions may be provided to the computer, for example, from a cloud-based network or from the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like. The computer 106 may include an input/output (I/O) interface to receive and/or send communication signals (data) to input and output devices, which may include a keyboard 104, a display 102, a mouse, a touch screen, touchless interface (e.g., a gesture recognition device) a printing device, a light pen, an optical storage device, a scanner, a microphone, a camera, a drive, communication cable and a network (either wired or wireless).

As it can be appreciated by persons skilled in the art, by using the novel hybrid OCT catheter disclosed herein the intravascular pressure of a lumen sample can be calculated without the need of swapping pressure sensing and imaging catheters. Advantageously, the PCI cost is reduced by using the same catheter for imaging and pressure measurements. Intravascular pressures can be calculated before and after the pullback (e.g., at the distal and proximal sides of stenosis). Moreover, the use of the same catheter for pressure measurement and imaging inside of a vascular sample (such as a vessel) reduces the PCI procedure time, reduces the cost of the procedure, and reduces the patient exposure to contrast agents, and can increase patient comfort.

### Other Embodiments, Modifications, or Combinations Thereof

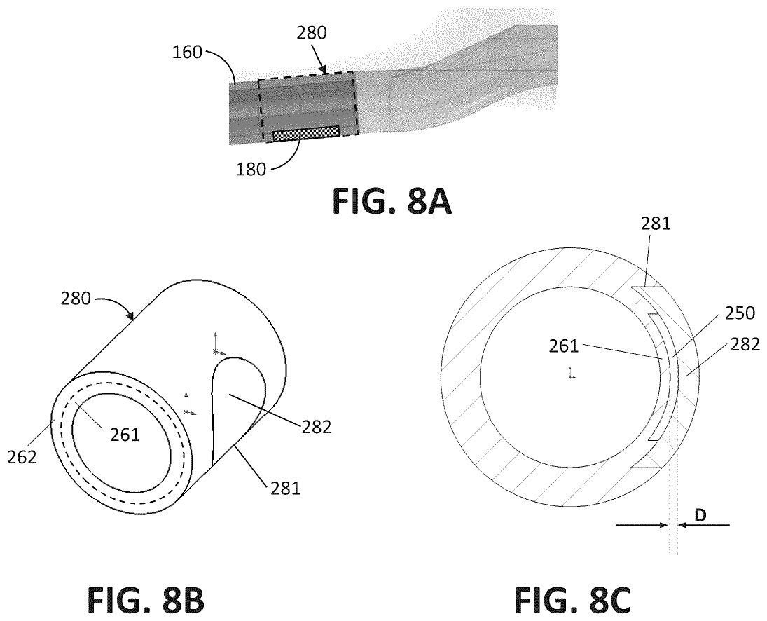

FIG. 8A, FIG. 8B, and FIG. 8C illustrate various views of another embodiment of the pressure sensor 180 useful for monitoring blood pressure and/or FFR in a vessel. FIG. 8A illustrates a portion of catheter 160 comprising a pressure sensor 180, according to this embodiment. As shown in FIG. 8A, a short cylindrical housing 280 is fixedly attached to the distal end of catheter 160, and the sensor 180 is incorporated into an outer surface of the housing 280. FIG. 8B shows a perspective view of the cylindrical housing 280. The housing 280 has an inner sheath 261, an outer sheath 262, and a side-opening 281 formed on the outer sheath 262. A flexible membrane 282 is arranged on the side-opening 281, so at to be embedded in the outer sheath 262 of the cylindrical housing 280.

FIG. 8C shows cross-section of the cylindrical housing 280. In this embodiment, the inner sheath 261 and the outer sheath 262 can be two components nested within each other, or can be a single component (e.g., a single sheath). However, when a single sheath is used for the housing, at least in the portion of the housing where the membrane 282 is embedded, the inner surface is equivalent to the inner sheath 161, and the outer surface is equivalent to the outer sheath 162 of the previous embodiment. That is, a wall of the housing 280 should have sufficient thickness to provide space for embedding the membrane 282 at a distance from the inner surface thereof, so as to form a small chamber 250 therebetween. Since the membrane 282 is embedded to be substantially tangential to the outer sheath 262, there is a built-in gap of distance D between an inner sheath 261 and the membrane 282. The gap between the inner surface 261 and the membrane 282 is sealed so as to create the small chamber 250 similar to the chamber 150 of the previous embodiment. When intravascular pressure is present, the membrane 282 is configured to deflect into the chamber 250 (towards) the inner surface 261 in response to intravascular pressure.

In this embodiment similar to the previous embodiment, a light beam emitted from the imaging core measures the distance D at the distal-most (park) position of the imaging core based on OCT principles, as explained above. At the park position, when light is transmitted from the imaging core, the light beam travels through the inner surface 261, through the chamber 250, and impinges on the flexible membrane 282. Light reflected or scattered by the membrane 282 and by the inner sheath 261 is collected by the imaging core and guided to the console 110 via the PIU 120. Therefore, similar to the other embodiments, the imaging catheter includes a chamber defined by the flexible membrane 282, a part of the inner sheath and a part of the outer sheath that are nested within each other, and the chamber provides an empty space into which the flexible membrane is deflected in response to the intravascular pressure. In the event that the membrane 282 breaks, the catheter can continue to be used for OCT imaging because the inner sheath 261 maintains the imaging core 168 sealed, and prevents any fluid of the vessel from entering into the imaging core.

In referring to the description, specific details are set forth in order to provide a thorough understanding of the examples disclosed. In other instances, well-known methods, procedures, components and circuits have not been described in detail as not to unnecessarily lengthen the present disclosure. Unless defined otherwise herein, all technical and scientific terms used herein have the same meaning as commonly understood by persons of ordinary skill in the art to which this disclosure belongs. In that regard, the scope of the present disclosure is not limited by the specification or drawings, but rather only by the broadest reasonable interpretation of the claim terms employed.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

Any patent, pre-grant patent publication, or other disclosure, in whole or in part, that is said to be incorporated by reference herein is incorporated only to the extent that the incorporated materials do not conflict with standard definitions or terms, or with statements and descriptions set forth in the present disclosure. As such, and to the extent necessary, the disclosure as explicitly set forth herein supersedes any conflicting material incorporated by reference.

What is claimed is:

1. An imaging catheter, comprising:
an imaging core, an inner sheath enclosing the imaging core, and an outer sheath surrounding the inner sheath;
a flexible membrane arranged on the outer sheath and configured to deflect in response to intravascular pressure,
wherein the outer sheath surrounds the inner sheath such that at least a part the inner sheath and a part of the outer sheath are nested within each other,
wherein the imaging catheter includes a chamber defined by the flexible membrane, the part of the inner sheath and the part of the outer sheath that are nested within each other, and
wherein the chamber provides an empty space into which the flexible membrane is deflected in response to the intravascular pressure.

2. The imaging catheter according to claim 1,
wherein the part of inner sheath and the part of the outer sheath that are nested within each other are coaxial to each other and at a predetermined distance therebetween, and
wherein the chamber has no fluid communication with the lumen of the inner sheath.

3. The imaging catheter according to claim 1,
wherein the flexible membrane is arranged on a side opening formed in a portion of the outer sheath;
wherein the imaging core is arranged inside the inner sheath,
wherein the outer sheath is arranged to coaxially overlap at least a distal portion of the inner sheath, and
wherein the chamber is formed by the space between the distal portion of the inner sheath overlapped by the portion of the outer sheath to which the flexible membrane is attached.

4. The imaging catheter according to claim 1,
wherein the flexible membrane is a circular silicone membrane held by a cylindrical frame,
wherein the cylindrical frame has a top flat surface with a circular opening and an arcuate bottom surface attached to an external surface of the outer sheath,
wherein the circular silicone membrane is arranged on the top flat surface substantially tangential to the external surface of the outer sheath, and
wherein the chamber includes the space between the inner sheath and the outer sheath, and the space between the silicon membrane and the inner sheath.

5. A system comprising:
an imaging catheter and a processor configured to acquire intravascular image data and intravascular pressure data from a vessel in a vasculature of a patient;
the imaging catheter comprising:
an imaging core, an inner sheath enclosing the imaging core, and an outer sheath surrounding the inner sheath; and
a flexible membrane arranged on the outer sheath and configured to deflect in response to intravascular pressure,
wherein the outer sheath surrounds the inner sheath such that at least a part the inner sheath and a part of the outer sheath are nested within each other,
wherein the imaging catheter includes a chamber defined by the flexible membrane, the part of the inner sheath and the part of the outer sheath that are nested within each other, and
wherein the chamber provides an empty space into which the flexible membrane is deflected in response to the intravascular pressure;
the processor configured to:
control the imaging core to scan the flexible membrane by transmitting a light beam through the inner sheath and the chamber, and
calculate the intravascular pressure within the vessel based upon light reflected or scattered by the flexible membrane and by the inner sheath.

6. The system according to claim 5,
wherein the part of inner sheath and the part of the outer sheath that are nested within each other are coaxial to each other and at a predetermined distance therebetween, and
wherein the chamber has no fluid communication with the lumen of the inner sheath or with fluids in the vessel.

7. The system according to claim 6,
wherein the flexible membrane is arranged on a side opening of the outer sheath;
wherein the imaging core is arranged inside the inner sheath and configured to transmit the light beam at an angle with respect to the longitudinal axis,
wherein the processor is operatively coupled to the imaging core and configured to:
control the imaging core to scan the flexible membrane with the light beam that is transmitted through the inner sheath, through the chamber, and through the side opening of the outer sheath,
calculate an amount of deflection of the flexible membrane based upon the light reflected or scattered by the flexible membrane and by the inner sheath; and
generate intravascular pressure data based on the calculated amount of deflection.

8. The system according to claim 6,
wherein the processor is further configured to:
calculate an amount of deflection of the flexible membrane based upon the light reflected or scattered by the flexible membrane and by the inner sheath; and
wherein the processor calculates the intravascular pressure based on the amount of deflection of the flexible membrane, and
wherein the amount of deflection is equal a difference between the predetermined distance between the inner sheath and the outer sheath and an average position of the flexible membrane deflected towards the inner sheath in response to the intravascular pressure.

9. The system according to claim 5,
wherein the processor is further configured to:
calculate the intravascular pressure at a first location distal to a stenosis and at second location proximal to the stenosis of the vessel; and
calculate a fractional flow reserve (FFR) based on the intravascular pressure calculated at the first and second locations.

10. The system according to claim 5,
wherein the flexible membrane is a circular silicon membrane, and
wherein the processor calculates the intravascular pressure according to the DiGiovanni elasticity equation, where pressure (P) is given by Equation (1)
$$P = [ \frac{16}{3 ( 1 - {μ}^{2} )} ( \frac{E {h}^{3}}{{r}^{4}} ) ] d + [ \frac{( 7 - μ )}{3 ( 1 - μ )} ( \frac{E h}{{r}^{4}} ) ] {d}^{3} , \qquad Eq . ( 1 )$$
where E is the Young's modulus, μ is the Poisson's ratio, r is the radius, h is the thickness, and d is an amount of deflection of the silicone membrane in response to the intravascular pressure.

11. The system according to claim 5,
wherein the processor is further configured to:
generate an OCT image based upon the light reflected or scattered by the flexible membrane and by the inner sheath.

12. The system according to claim 11,
wherein the processor is further configured to:
calculate an amount of deflection of the flexible membrane based upon peak signals in the OCT image corresponding to the light reflected or scattered by the flexible membrane and by the inner sheath, and
wherein the amount of deflection is proportional to a distance between a first signal shown in the OCT image corresponding to light reflected or scattered by the inner sheath and a second signal shown in the OCT image corresponding to an average of the light reflected or scattered by the flexible membrane deflected towards the inner sheath in response to the intravascular pressure.

13. The system according to claim 5,
wherein the processor is further configured to:
control rotation and pullback of the imaging core such that the imaging core first irradiates the flexible membrane by transmitting the light beam through the inner sheath and through the chamber, and subsequently scans the vessel by transmitting the light beam only through the inner sheath.

14. The system according to claim 5,
wherein the processor controls the imaging core to irradiate the flexible membrane and the inner sheath with the light beam while rotating the imaging core prior to initiating a pullback, and subsequently controls the imaging core to scan the vessel wall with the light beam in a helicoidally oriented path while the imaging core is rotated and pullback.

15. The system according to claim 5,
wherein the processor is further configured to:
generate a first OCT image based upon the light reflected or scattered by the flexible membrane and by the inner sheath while the imaging core is rotated without being pullback, and
generate a second OCT image based upon light reflected or scattered by the vessel wall while the imaging core is rotated and pullback.

16. The system according to claim 5,
wherein the flexible membrane is a circular silicone membrane held by a substantially cylindrical frame,
wherein the cylindrical frame has an arcuate bottom surface attached to the outer sheath and a flat surface with an opening, and
wherein the circular silicone membrane is arranged on the flat surface substantially tangential to an external surface of the outer sheath.

17. A method for simultaneously acquiring intravascular image data and intravascular pressure data, the method comprising:
inserting an imaging catheter into a vessel of a patient's vasculature, the imaging catheter comprising an outer sheath having lumen along a longitudinal axis and a flexible membrane arranged on a side opening of the outer sheath perpendicular to the longitudinal axis, an inner sheath inserted into the lumen of the outer sheath such that the inner sheath and the outer sheath are coaxial to each other and at a predetermined distance therebetween, and an imaging core arranged inside the inner sheath and configured to transmit a light beam at an angle with respect to the longitudinal axis;
controlling, using a processor operatively coupled to the imaging core, the imaging core arranged inside the inner sheath to scan the flexible membrane with a light beam that is transmitted through the inner sheath and through the side opening of the outer sheath at an angle with respect to the longitudinal axis; and
a processor operatively coupled to the imaging core and configured to:
calculate intravascular pressure based upon light reflected or scattered by the flexible membrane and by the inner sheath.
controlling the imaging core to irradiate the pressure-sensing membrane and a vessel wall of the vessel with a light beam;
receiving a pressure measurement signal from light reflected or scattered by the pressure-sensing membrane and by the inner sheath;
receiving an image signal from light reflected or scattered by the vessel while the imaging core is rotated and/or pullback with respect to the inner sheath and the outer sheath;
generating an image of the vessel based on the image signal; and
calculating a pressure parameter based on the pressure measurement signal.

18. The method of claim 17, further comprising:
outputting, to a display device, the image of the vessel and the calculated pressure parameter.

19. The method according to claim 17,
wherein the calculating a pressure parameter includes:
calculating the intravascular pressure at a first location distal to a stenosis and at second location proximal to the stenosis of the vessel; and
calculating a fractional flow reserve (FFR) based on the intravascular pressure calculated at the first and second locations.

20. The method according to claim 17, further comprising:
generating, using the processor, a first OCT image based upon the light reflected or scattered by the flexible membrane and by the inner sheath while the imaging core is rotated without being pullback, and
generating, using the processor, a second OCT image based upon light reflected or scattered by the vessel wall while the imaging core is rotated and pullback.
